(19) United States
(12) Patent Application Publication
(10) Pub. No.: US 2010/0298845 A1
(43) Pub. Date: Nov. 25, 2010

(54) REMOTE MANIPULATOR DEVICE

(76) Inventors: Brian L. Kidd, St. Louis, MO (US); Nathan Kastelein, St. Louis, MO (US)

Correspondence Address: HARNESS, DICKEY, & PIERCE, P.L.C, 7700 Bonhomme, Suite 400, ST. LOUIS, MO, 63105, US

(21) Appl. No.: 12/786,833

(22) Filed: May 25, 2010

Related U.S. Application Data
(60) Provisional application No. 61/180,926, filed on May 25, 2009.

(51) Int. Cl. A61B 19/00

(52) U.S. Cl. 606/130

(57) ABSTRACT

A system for operating a catheter having a distal end adapted to be navigated in the body, and a proximal end having a handle with a translatable control and a rotatable control for acting on the distal end of the device includes a support for receiving and engaging the handle of the catheter; a translation mechanism for advancing and retracting the support to advance and retract a catheter whose handle is received in the support; a rotation mechanism for rotating the support to rotate a catheter whose handle is received in the support; a translation operator for engaging the translatable control of a catheter whose handle is received in the support and operating the translatable control to act on the distal end of the device; and a rotation operator for engaging the rotatable control of a catheter whose handle is received in the support and operating the rotatable control to act on the distal end of the device.

### CROSS-REFERENCE TO RELATED APPLICATIONS

This application claims priority to prior U.S. Provisional Patent Application No. 61/180,926 filed on May 25, 2009. The entire disclosure of the above application is incorporated herein by reference.

### BACKGROUND

This section provides background information related to the present disclosure which is not necessarily prior art.

This invention relates to automating the operation of medical devices.

Significant progress has been made in automating the navigation of medical devices in the body. Remote navigation systems, such as the Niobe® magnetic navigation system available from Stereotaxis, Inc., St. Louis, Mo., allows a physician to remotely orient the distal end of a medical device in the body. More recently, an automated advancer for advancing and retracting the device in the body has also become available, allowing more fully automated catheter navigation systems. However, a practical means of completely automating (under the supervision of a physician) the operation of medical devices, whereby a medical device can be automatically navigated to a particular location and then operated to perform some diagnostic or therapeutic procedure has not been available. This is particularly true with respect to automating the operation of conventional manually operated medical devices.

### SUMMARY

This section provides a general summary of the disclosure, and is not a comprehensive disclosure of its full scope or all of its features.

Embodiments of the present invention provide a remote manipulator that not only can manipulate a conventional catheter, but can operate its controls. This not only allows the catheter to be remotely navigated, but also to be remotely operated. This allows a physician to conduct the procedure away from the patient, and also permits the complete automation of the procedure, with a computer navigating the catheter and operating the catheter without the need for human intervention.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a front perspective view of a preferred embodiment of a remote manipulator device in accordance with the principles of this invention;

FIG. 2 is a rear perspective view of the preferred embodiment of the remote manipulator device, with the drive cables removed for clarity;

FIG. 3 is a top plan view of the preferred embodiment of the remote manipulator device, with the drive cables removed for clarity;

FIG. 4 is a rear elevation view of the preferred embodiment of the remote manipulator device, with the drive cables removed for clarity;

FIG. 5 is a left side elevation view of the preferred embodiment of the remote manipulator device, with the drive cables removed for clarity;

FIG. 6 is an exploded view of the preferred embodiment of the remote manipulator device;

FIG. 7 is a front perspective view of the preferred embodiment of the remote manipulator device, with the device driver removed for clarity;

FIG. 8 is a rear perspective view of the preferred embodiment of the remote manipulator device, with the device driver removed for clarity;

FIG. 9 is a left side elevation view of the preferred embodiment of the remote manipulator device, with the device driver removed for clarity;

FIG. 10 is a top plan view of the preferred embodiment of the remote manipulator device, with the device driver removed for clarity;

FIG. 11 is a front elevation view of the preferred embodiment of the remote manipulator device, with the device driver removed for clarity;

FIG. 12 is a rear perspective view of the preferred embodiment, with the cover removed from the controller;

FIG. 13 is a perspective view of the device driver of the preferred embodiment;

FIG. 14 is a side elevation view of the device driver of the preferred embodiment;

FIG. 15 is an front end elevation view of the device driver of the preferred embodiment;

FIG. 16 is a rear end elevation view of the device driver of the preferred embodiment;

FIG. 17 is a top plan view of the device driver of the preferred embodiment;

FIG. 18 is a perspective view of the body of the device driver, with the upper and lower members separated to show the details of construction;

FIG. 19 is a perspective view of the body of the device driver, with the upper member removed to show the details of construction;

FIG. 20 is an exploded perspective view of the body of the device driver, with the upper member removed;

FIG. 21 is an exploded perspective view of the body of the device driver, with parts removed to show details of construction;

FIG. 22 is a perspective view of the device interface of the preferred embodiment;

FIG. 23 is a top plan view of the device interface of the preferred embodiment;

FIG. 24 is a side elevation view of the device interface of the preferred embodiment;

FIG. 25 is a bottom plan view of the device interface of the preferred embodiment;

FIG. 26 is an exploded perspective view of the device interface of the preferred embodiment;

FIG. 27 is a perspective view of one of the clamps used in the device interface of the preferred embodiment;

FIG. 28 is a rear elevation view of the clamp;

FIG. 29 is a front elevation view of the clamp;

FIG. 30 is a top plan view of the clamp;

FIG. 31 is a side elevation view of the clamp, showing the hinged connection between the base and cover;

FIG. 32 is an exploded perspective view of the clamp;

FIG. 33 is a perspective view of the split ring adapter used in the clamp;

FIG. 34 is a front elevation view of the split ring adapter;

FIG. 35 is a side elevation view of the split ring adapter;

FIG. 36 is an exploded perspective view of the split ring adapter;

FIG. 37 is a perspective view showing the support installed on the bed;

FIG. 38 is a perspective view showing the mounting of the bracket and controller on the base plate of the support;

FIG. 39 is a perspective view of the proximal end of a telescoping catheter support;

FIG. 40A is a perspective view of a medical device being placed in the clamps;

FIG. 40B is a perspective view of the covers of the clamps being closed over a medical device being placed in the clamps; and

FIG. 40C is a perspective view of the covers of the clamps being latched to secure a medical device being placed in the clamps.

Corresponding reference numerals indicate corresponding parts throughout the several views of the drawings.

### DETAILED DESCRIPTION

Example embodiments will now be described more fully with reference to the accompanying drawings.

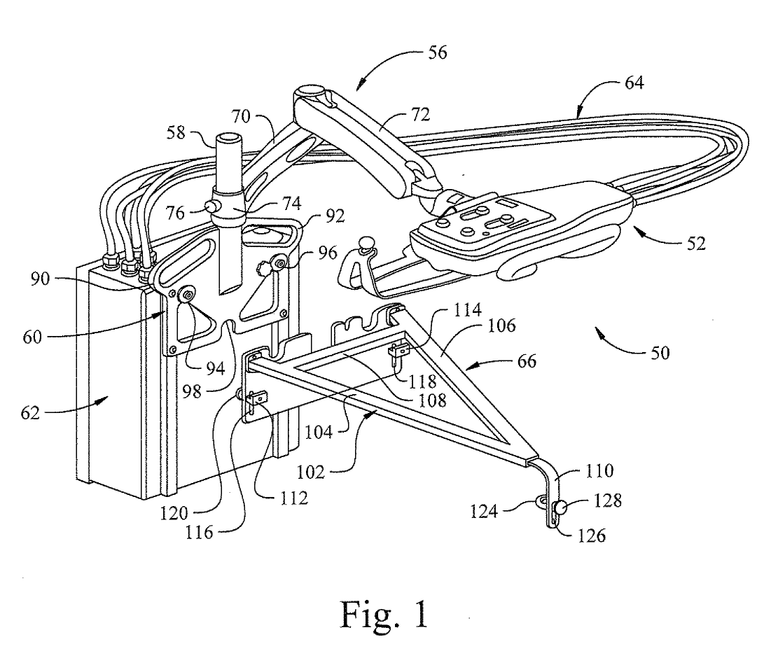

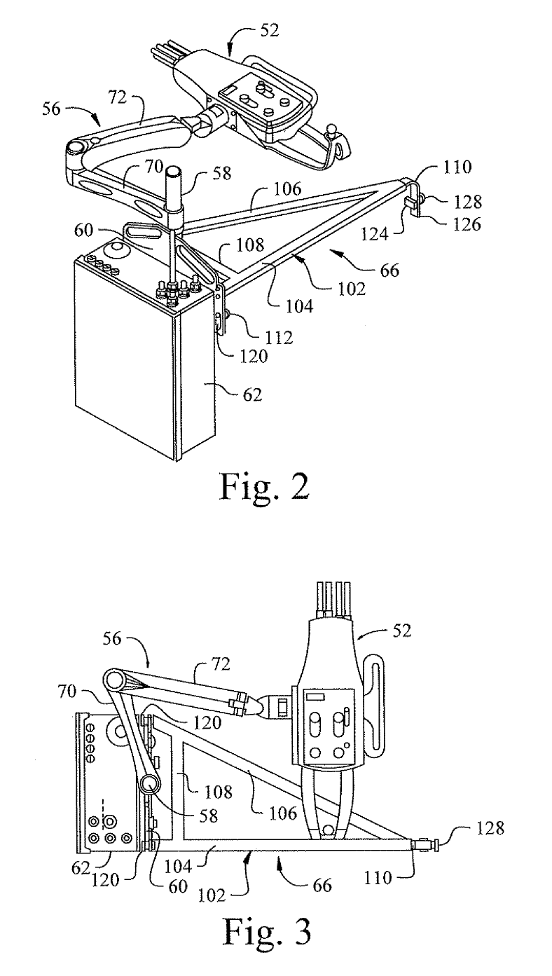

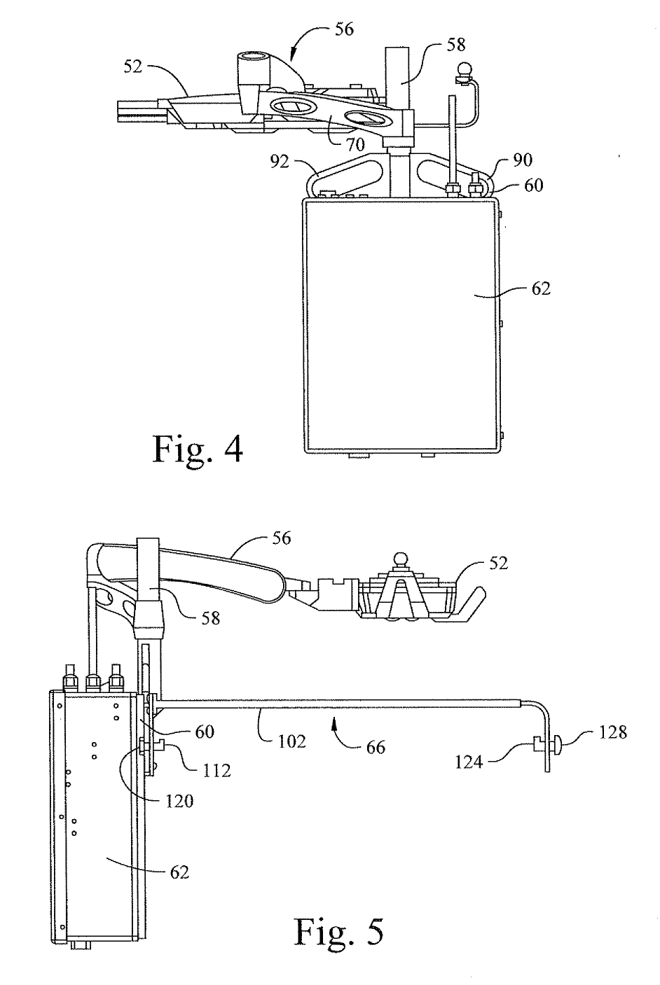

A preferred embodiment of a remote manipulator device constructed according to the principles of this invention is indicated generally as 50 in FIGS. 1-5. The remote manipulator 50 is adapted to engage and operate a medical device. The medical device may be, for example, an electrophysiology catheter of the type comprising an elongate sheath having a handle at its proximal end, and an electrophysiology wire extending through the sheath and out the distal end, forming a loop. The handle preferably has controls for manipulating the distal end of the wire, for example a translatable control for bending the distal end, and a rotatable control for expanding and contracting the loop. While this preferred embodiment is described with respect to an electrophysiology catheter, this invention is not limited to electrophysiology catheters and applies to any medical device with an elongate portion, and a handle at the proximal end that can be manipulated to position and operate the distal end of the elongate portion of the medical device.

As shown in the FIGS. 1-5, the remote manipulator 50 comprises a device driver 52 for mounting and operating a device interface 54 (not shown in FIGS. 1-5), which in this preferred embodiment is both replaceable and disposable. The device driver 52 is mounted on the distal end of an articulated arm 56, whose proximal end is mounted on a post 58 on bracket 60. A controller 62, which can be mounted to the bracket 60, is connected to the device driver 52 by a plurality of flexible drive cables 64 to operate the device driver 52. A platform 66, attachable to the patient bed (not shown in FIGS. 1-5), can mount the bracket 60 and/or the controller 62.

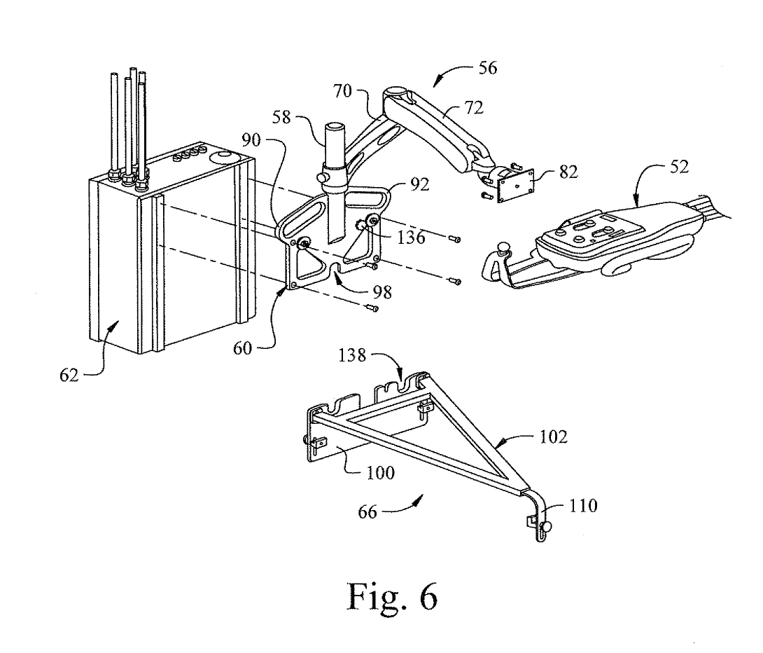

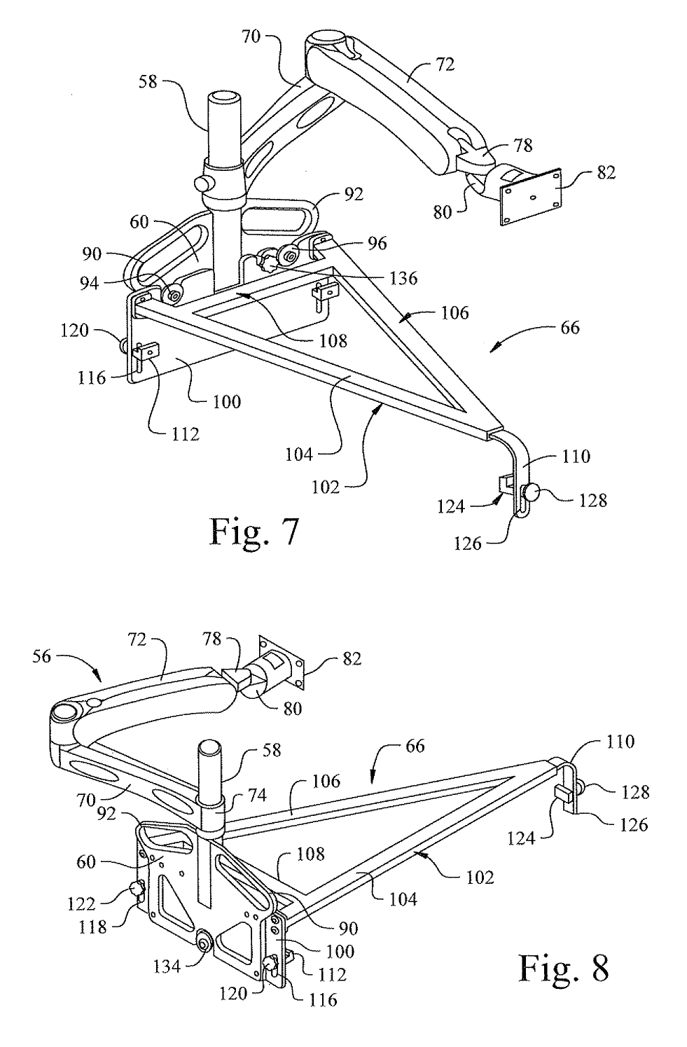

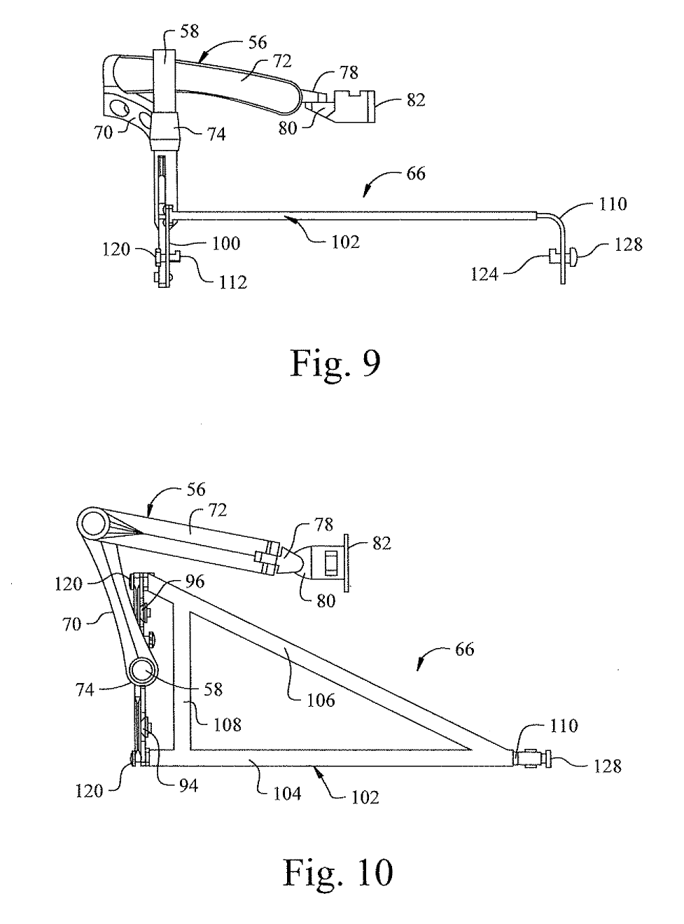

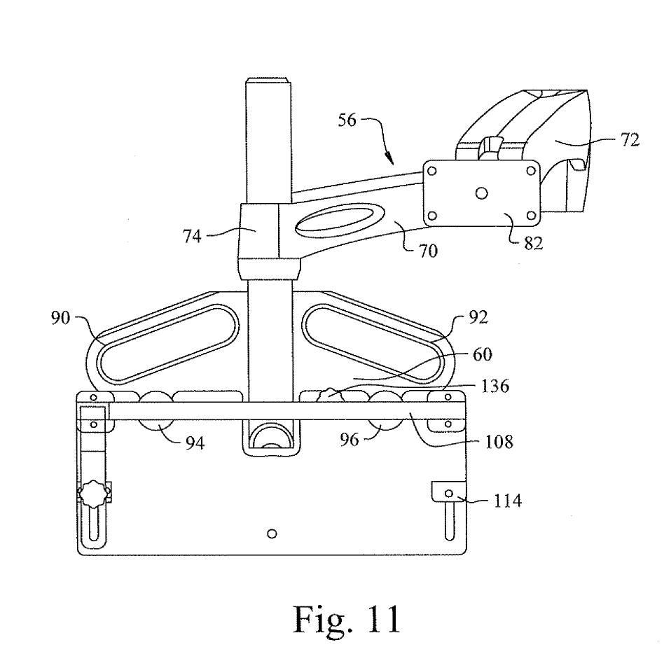

As shown in FIG. 6-11, the articulated arm 56 comprises proximal and distal sections 70 and 72. Proximal section 70 has a collar 74 at its proximal end for mounting the proximal section 70 on the post 58. The proximal section 70 can translate up and down, and rotate around, the post 58. A locking screw 76 allows the collar and thus, the proximal section 70 to be releasably locked relative to the post 58. The distal end of the proximal section 70 is pivotally connected to the proximal end of the distal section 72. A lock (not shown) can be provided to releasably lock the proximal and distal sections 70 and 72 relative to each other. A first wrist element 78 is pivotally mounted to the distal end of the distal section 72 to pivot about a first axis generally perpendicular to the longitudinal axis of the distal section. A second wrist element 80 is pivotally mounted to the first wrist element to pivot about a second axis generally perpendicular to the first axis. A mounting plate 82 is rotatably mounted to the second wrist element 80 to rotate about a third axis, perpendicular to the first axis. The wrists pivoting about the first and second axis and the mounting plate rotating about the third axis are selectively releasably lockable, to releasably secure the articulated arm in a desired configuration with the device driver 52 in a desired location.

As also shown in FIGS. 6-11, the bracket 60 has hand loops 90 and 92 thereon. Pins 94 and 96 with enlarged conical heads project from the front face of the bracket 60. There is a semi-circular notch 98 in the bottom of the bracket 60.

As also shown in FIGS. 6-11, the platform 66 comprises a base plate 100, and a generally right-triangular frame 102, comprising legs 104 and 106, extending from the base plate 100 and leg 108 extending between the legs 104 and 106. A generally L-shaped bracket 110 telescopes from the leg 104 of the frame 102. Cleats 112 and 114 are mounted in slots 116 and 118 in the base plate 100 with threaded bolts 120 and 122 which have knurled heads that allow the cleats to be tightened against the side rail typically found on a patient bed. Similarly, a cleat 124 is mounted in slot 126 in the end of L-shaped bracket 110 with a threaded bolt 128 with a knurled head that allows the cleat to be tightened against the side rail of the patient bed. The top edge of the base plate 100 has semi-circular notches 130 and 132 for engaging the pins 94 and 96 on the bracket, and the back face of the base plate has a projecting pin 134 for engaging the semi-circular notch 98 in the bracket 60. A bolt 136 on the bracket 60 can be tightened to engage a slot 138 on the base plate 100 to secure the bracket and base plate 100.

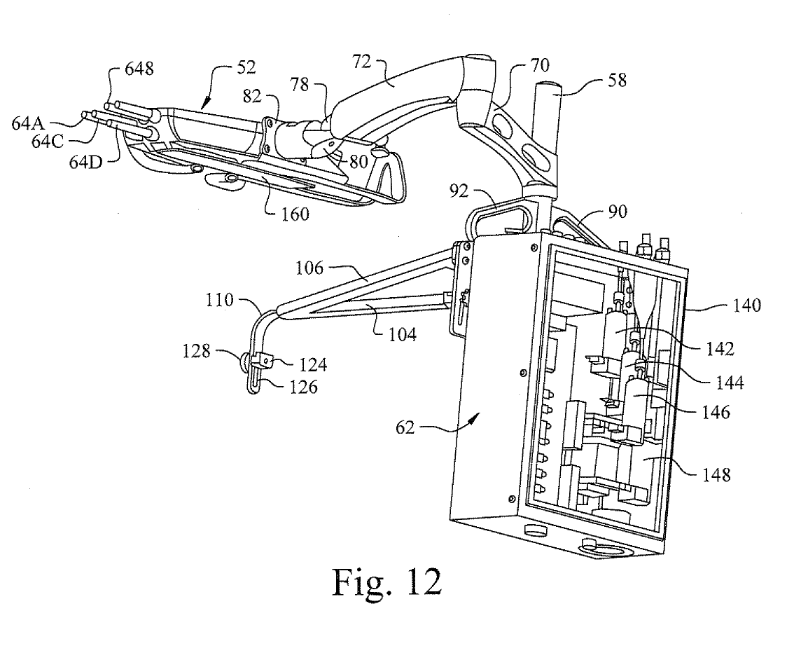

The controller 62 can be attached to the bracket 60 and mounted on the platform 66. As shown in FIG. 12, the controller includes a housing 140 with four drives: a drive 142 for operating a first clamp driver on the device driver 52; a drive 144 for operating the second clamp translation driver on the device driver; a drive 146 for operating a second clamp driver on the device driver; and a drive 148 for operating the translation driver on the device driver for translating the device, as will be described in more detail below. Flexible drive cables 64 connect each drive in the controller with its respective driver in the device drive 52.

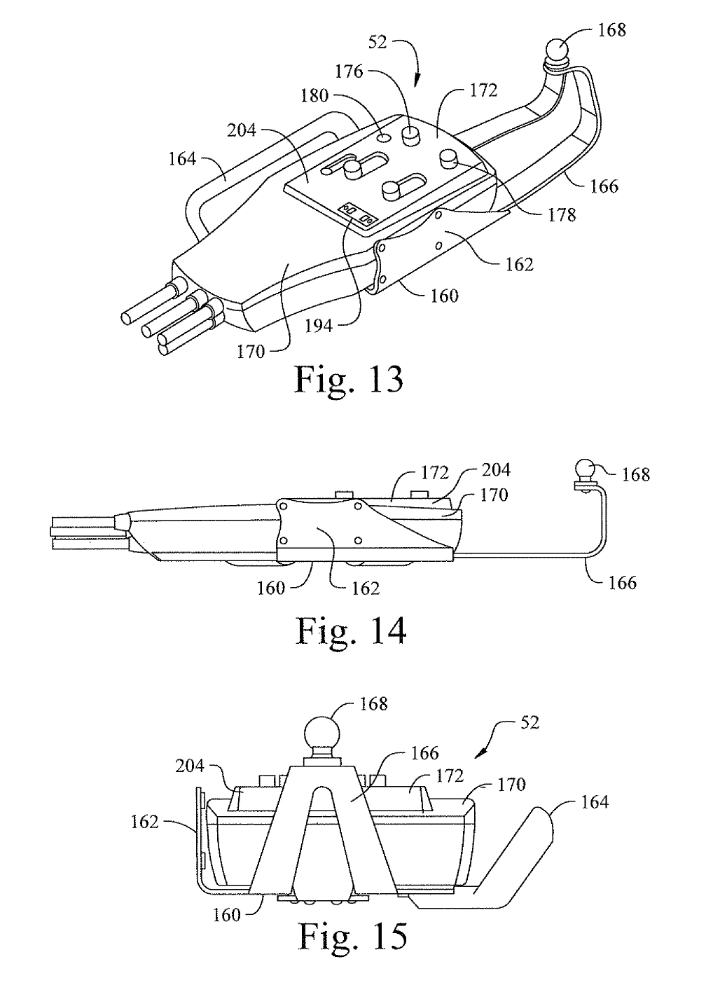

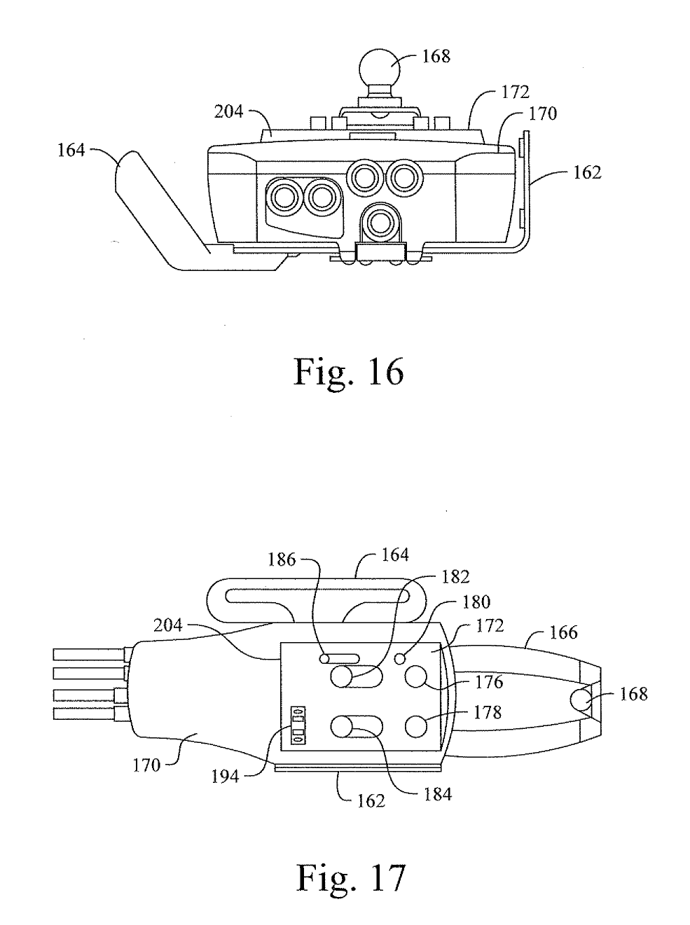

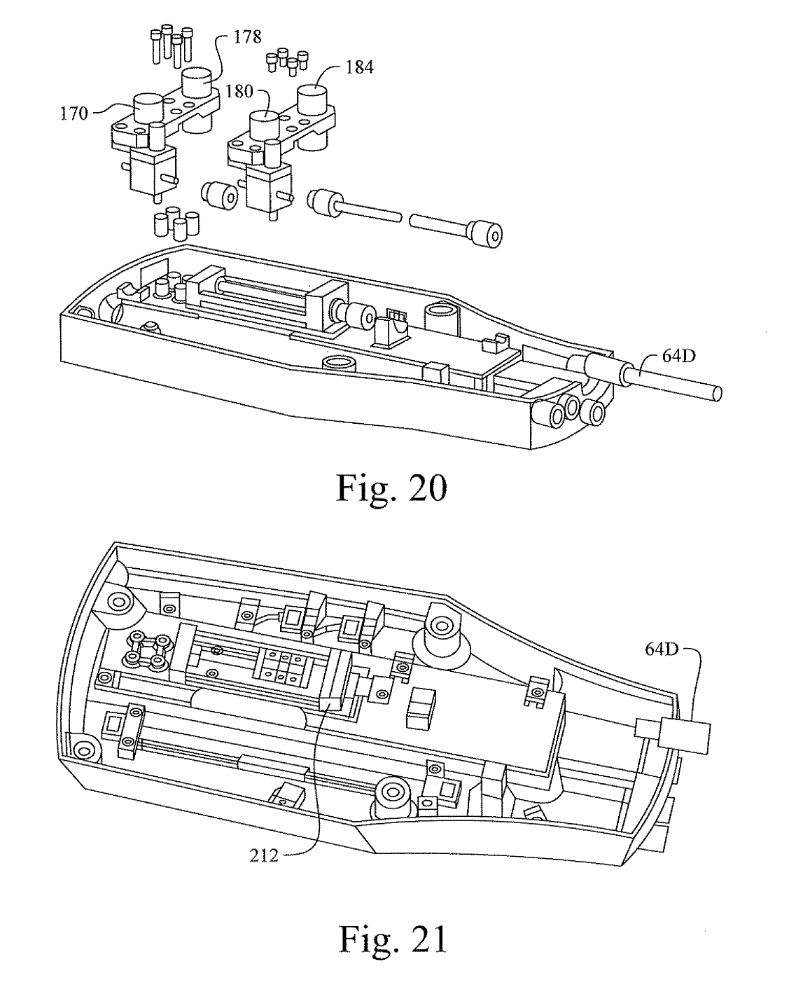

As shown in FIGS. 13-21, the device driver 52 comprises a base 160 having a mount 162 for attaching the device driver to the mounting plate 82 on the end of the articulated arm 56. A hand loop 164 on the opposite side of the base 160 from the mount 162 can be provided for gripping the device driver 52 to reposition it. A generally V-shaped bracket 166 projects from the front of the base 160, the end of the bracket is bent upwardly and has a fixture 168 mounted thereon for mounting a sheath, as described in more detail below.

The device driver 52 further comprises a body 170 mounted on the base 160 to translate forwardly and rearwardly with respect to the base.

The top surface of the body 170 has a platform 172 for mounting a device interface 54 thereon. As described below, the device interface 54 has one or more clamps 174 (not shown in FIGS. 13-21) for engaging and operating the handle of an elongate medical device. The platform 172 has a first pair of mounting sockets 176 and 178 for engaging a clamp 174 on the device interface 54, and a drive socket 180 for receiving and engaging a drive shaft of the clamp to operate the clamp. In this preferred embodiment, the platform 172 also has a second pair of mounting sockets 182 and 184 for engaging a second clamp 174 on the device interface 54, and a drive socket 186 for receiving and engaging a drive shaft of the second clamp to operate the second clamp. The second pair of sockets 182 and 184, and the drive socket 186 are disposed in slots so that they can translate relative to the first set of sockets 176 and 178, to accommodate relative movement between the clamps 174 on the device interface 54, as will be described in more detail below. There is an electrical contact pad 194 on the platform 172 for making electrical contact with a corresponding electrical contact pad 196 on the device interface 54.

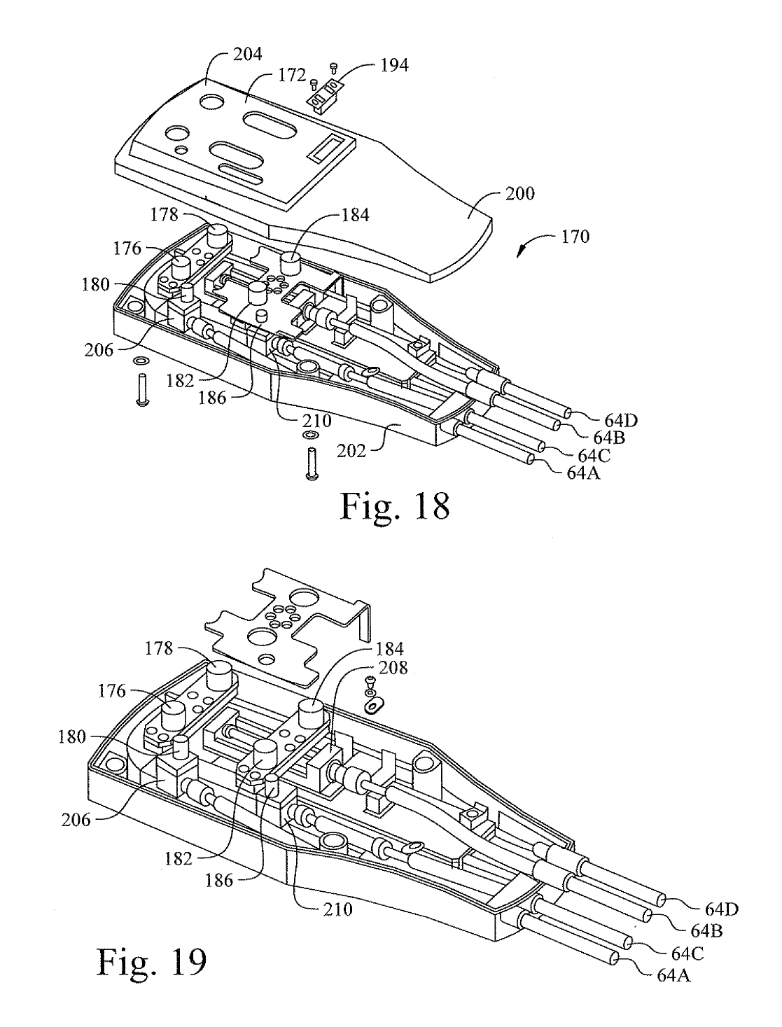

As shown in detail in FIG. 18, the body 170 comprises upper and lower housing members 200 and 202. The platform 172 is formed in the top of the upper housing member 200, and comprises a raised platform area 204 with openings for the sockets 176 and 178 and 182 and 184, as well as contact pad 194. As shown in FIG. 19, in the lower housing member 202, the distal end of flexible drive cable 64A, connects the drive 142 to a transmission 206 for turning socket 180 to operate a first clamp 174 on a device interface 54 seated on the platform 172. The distal end of a cable 64B connects the drive 144 to a screw-driven translation mechanism 208 for translating the second pair of sockets 182 and 184 relative to first pair or sockets 176 and 178. The distal end of cable 64C connects the drive 146 to a transmission 210 for turning socket 186 to operate a second clamp 174 on a device interface 54. Lastly, a flexible drive cable 64D connects the drive 148 to a screw translation mechanism 212 (FIG. 21) for moving the body 170 relative to the base 160.

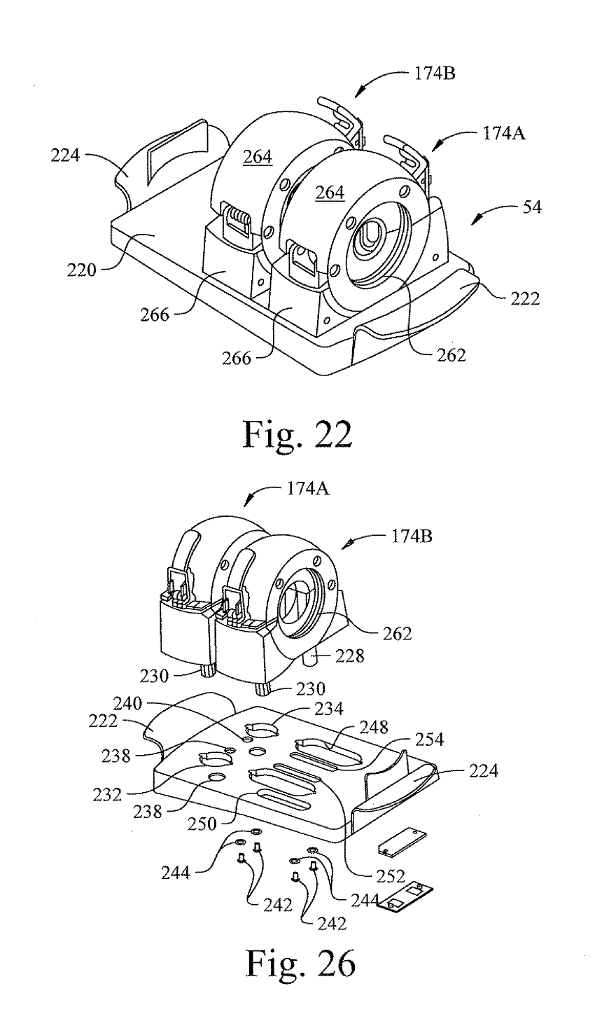

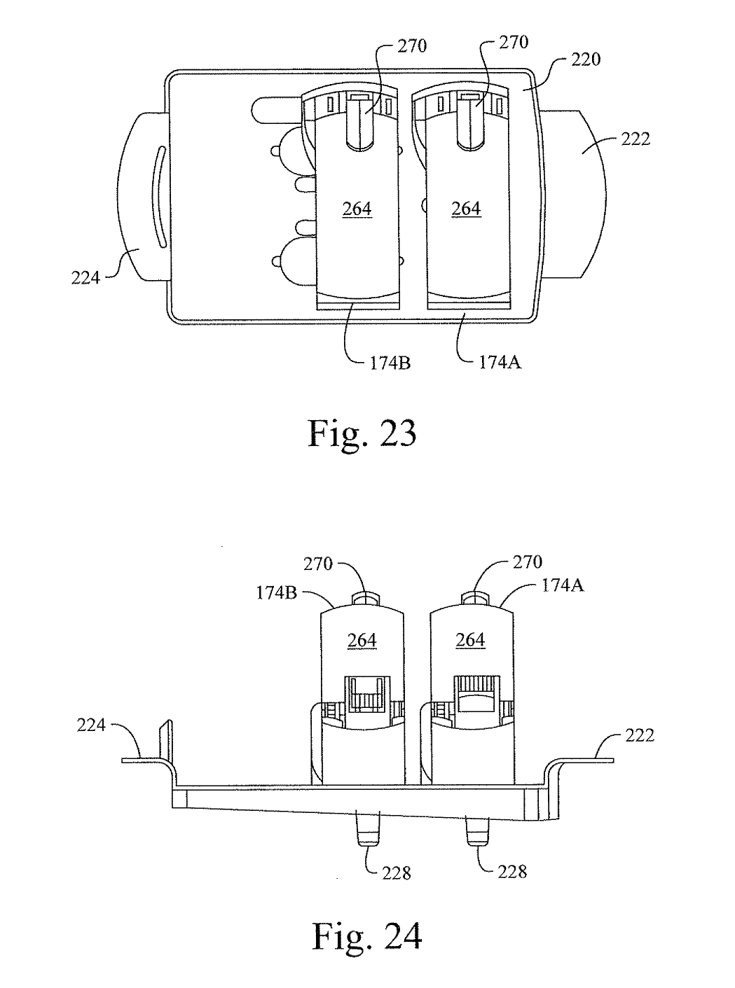

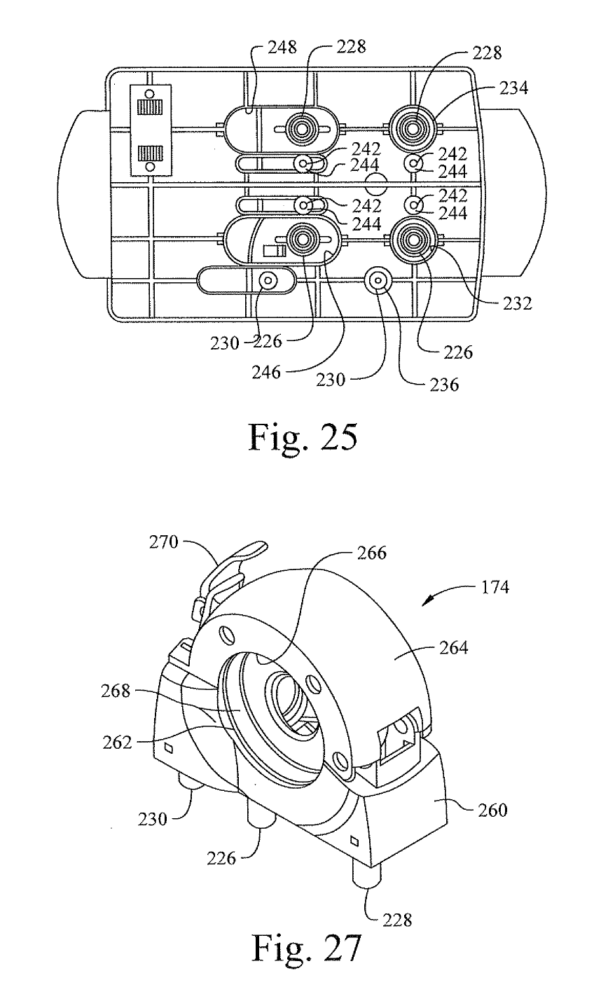

As shown in FIGS. 22-26, the device interface 54 comprises a tray 220 that is adapted to fit onto the raised platform 204 forming the platform 172 on the device driver 52. The tray 220 has a generally rectangular shape, but preferably has a curved side or other feature that prevents the tray from being installed on the device driver 52 incorrectly. There are handles 222 and 224 at either end of the tray 220 to facilitate handling the tray. One or more clamps 174 are mounted on the tray 220. In this preferred embodiment there are two clamps, a first clamp 174A, which is fixedly mounted with respect to the tray 220, and a second clamp 174B, which is slidably mounted with respect to the tray 220. As described in more detail below, each of the clamps has two depending pins 226 and 228, and a depending drive spline 230. The tray 220 has a pair of holes 232 and 234 for receiving the pins 226 and 228 of clamp 174A, and a hole 236 for receiving the drive spline 230. Two mounting holes 238 and 240 allow the clamp 174A to be secured to the tray with screws 242 and washers 244. The tray 220 also has a pair of slots 246 and 248 for receiving the pins 226 and 228 of clamp 174B, and a slot 250 for receiving the drive spline 230. Two mounding slots 252 and 254 allow the clamp 174B to be secured to the tray with screws 242 and washers 244. The slots 246, 248, 250 and 252 and 254 allow the clamp to translate on the tray 220. A contact plate 192 is secured on the bottom of the tray for making electrical contact with the contact pad 194 on the device driver 52.

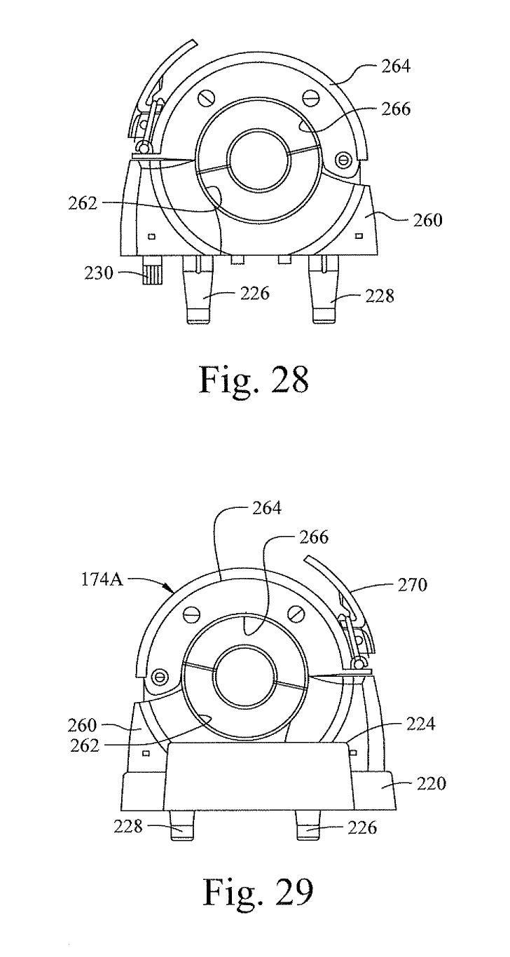

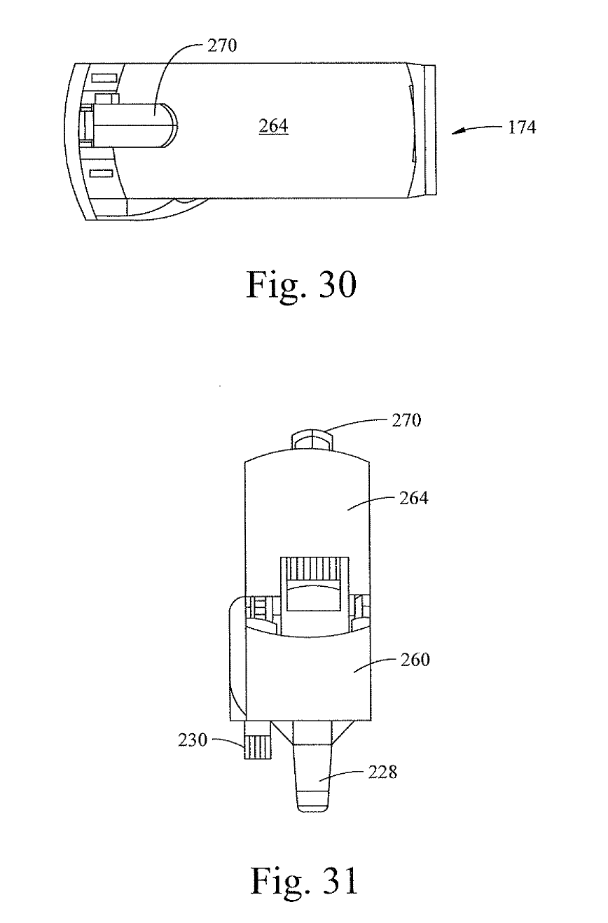

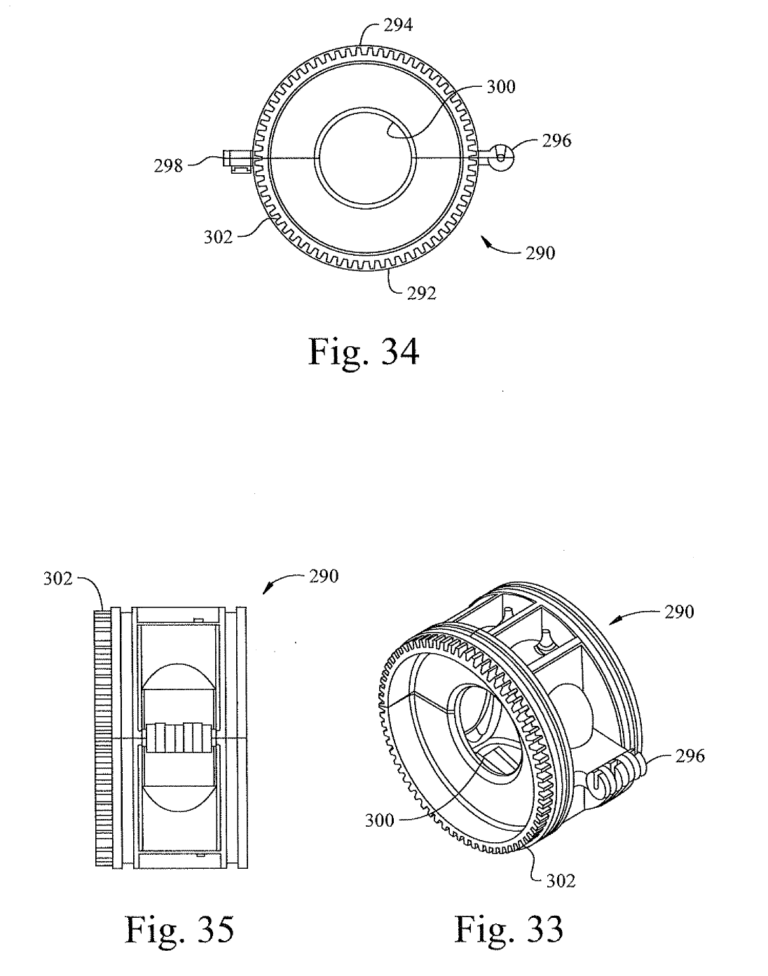

As shown in FIGS. 27-36, each of the clamps 174 comprises a base 260 which carries the pins 226 and 228 and the drive spline 230. The base 260 has a semi-circular notch 262. A generally arcuate cover 264 has a semi-circular notch 266 therein which is hingedly attached at one end to the base 260, to pivot between an open position and closed position, in which the semi-circular notch 262 in the base 260 and the semi-circular notch 266 in the cover 264 form a generally circular opening 268 through the clamp. An over-center latch 270 releasably secures the other end of the cover 264 to the base 260 to retain the cover in its closed configuration.

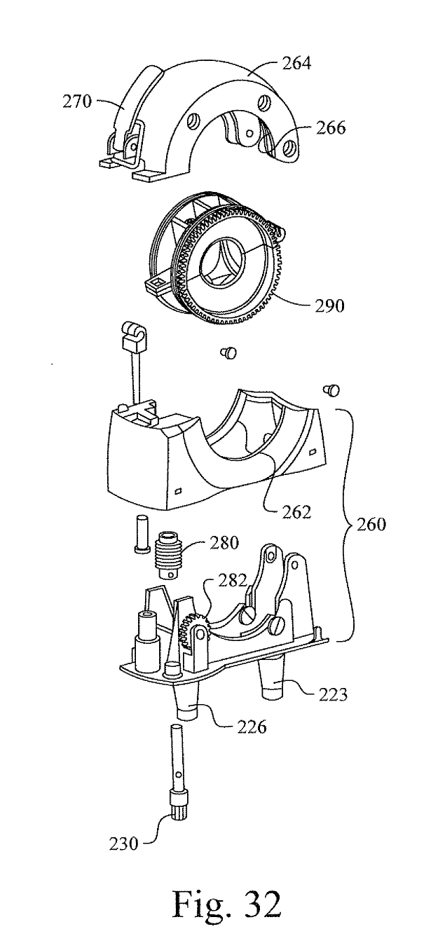

As best shown in FIG. 32, a worm gear 280 is mounted on the end of the spline 230 to rotate with the spline. The worm gear engages a sprocket 282, so that the sprocket turns when the spline 230 turns.

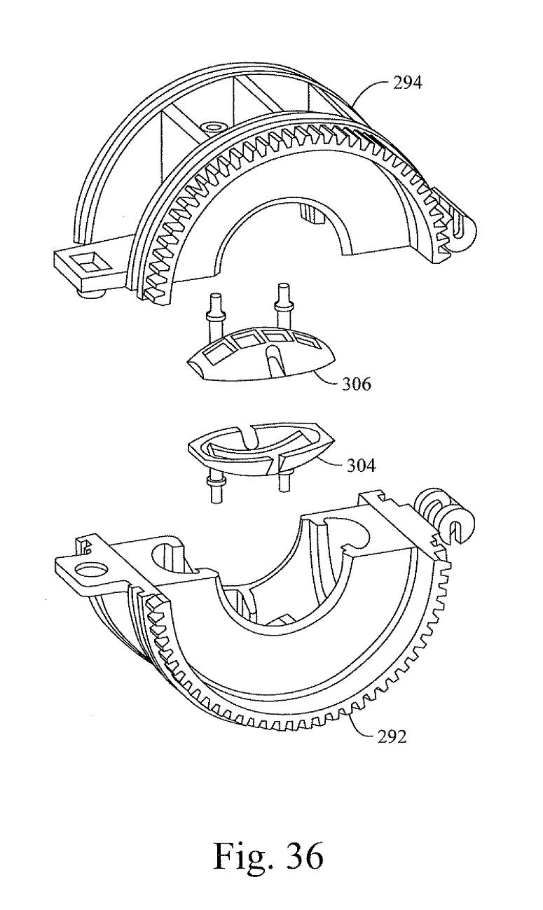

A split adapter ring 290 comprises first and second generally semi-circular halves 292 and 294, and has a hinged connection 296 at one side to pivot between an open position and a closed position in which the halves 292 and 294 form a ring. The other ends of the halves 292 and 294 are releasably connected, for example with a snap latch 298 to form a ring with a central opening 300. The split adapter ring 290 can be secured around a portion of the handle of a conventional manually operated medical device. The split adapter ring 290 has an associated ring gear 302 and is adapted to fit inside the clamp 174B, with the ring gear 302 engaged with the sprocket 282. The interior of each of the halves 292 and 294 can be specially adapted to receive the handle of a particular medical device, or various inserts (for example inserts 304 and 306 shown in FIG. 36) can be used to adapt a standardized halves to accommodate different medical devices.

### OPERATION

The operation of the preferred embodiment will be described with respect to a loop type EP Catheter, although, the invention is not so limited and embodiments of the remote manipulator can be used to operate a wide variety of medical devices which can be controlled through the manipulation of handle. The medical device has a handle with an actuator ring. Rotation of the actuator ring relative to the remainder of the handle causes the distal end of the loop catheter to bend. Translation of the actuator ring relative to the remainder of the handle causes the ring at the distal end to increase or decrease in size.

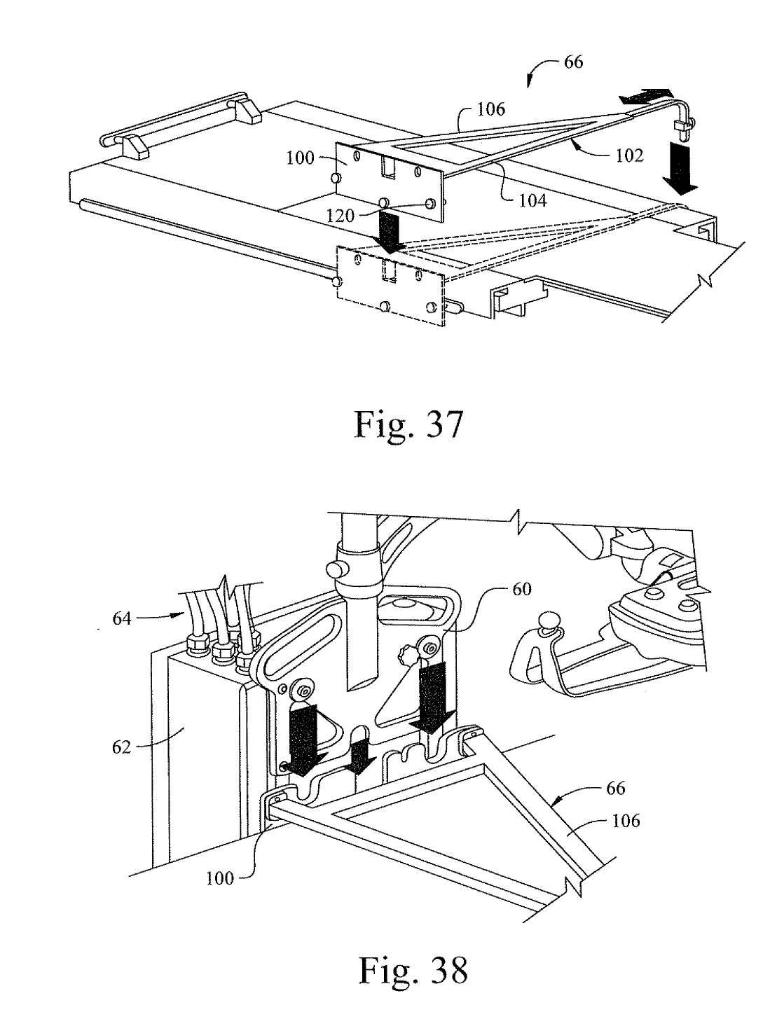

The system is first installed on a patient bed. As shown in FIG. 37, the platform 66 is laid across the surface of the bed, and secured to rails found on the sides of a typical patient bed. The cleats on the 112 and 114 on the base plate 100 are secured to the rail on one side of the bed by tightening the bolts 120 and 122. Similarly, on the opposite side of the bed, the cleat 124 on L-shaped bracket 110 is secured to the rail by tightening the bolt 128. Once the platform 66 has been secured on the patient bed, the bracket 60 is mounted on the platform 66. As shown in FIG. 38, the pins 94 and 96 on the bracket 60 fit into the semi-circular notches 130 and 132 in the support plate 100, and the pin 134 on the plate 100 engages the semi-circular notch 98 in the bracket 60. The bracket 60 and the support plate are secured by tightening bolt 136 on the bracket to engage the slot 138 in the base plate.

The device driver 52 is then positioned in the appropriate location by pivoting the articulating arm 56 around post 58, and securing it with bolt 76. The sections 70 and 72 can be moved and driver device 52 can be pivoted about the first, second, and third axes to bring the driver device to an appropriate position for conducting the procedure.

A surgical drape (not shown) in the form of an elongate plastic bag can be installed over the device driver 52 and articulating arm 56. The drape preferably has a puncturable window that generally corresponds in size and shape to the platform 204, and is aligned therewith. A replaceable disposable device interface 54, which can be provided in a sterile package, is removed from its sterile packaging and installed on the platform 204 of the device driver 52, with the pins 226 and 228 and spline 230 of each of the clamps 174 piercing the drape to connect to the sockets in the driver device. Alternatively, the window in the drape can be a framed opening, the tray 220 can seal with the frame, and the pins 226 and 228, and the spline 230 can engage their respective sockets without interference from the drape.

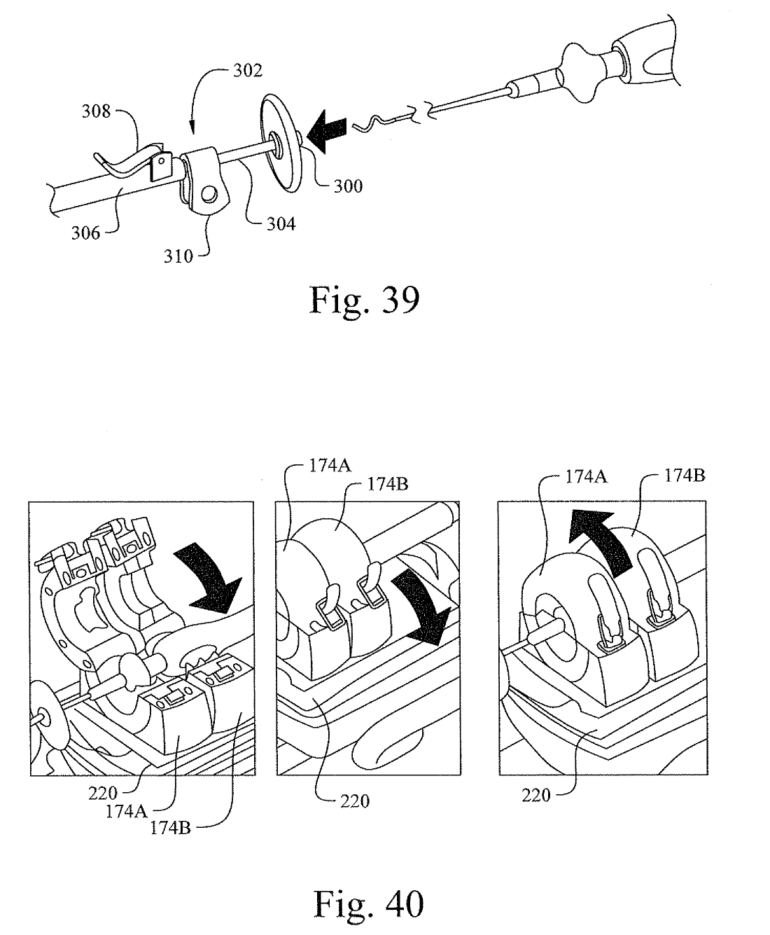

The elongate medical device is then prepared for use and mounting in the remote manipulator system. As shown in FIG. 39, the distal end of the medical device is introduced into the opening 300 in the proximal end of a telescoping catheter support 302. The telescoping catheter support 302 comprises at least two relatively telescoping tubes 304 and 306 with a lock 308 for locking the tubes 304 and 306 in position to set the length of the support. The distal end of the support 302 engages the introducer sheath. A clip 310 allows the proximal end of the catheter support 302 to engage the mount 168.

As shown in FIGS. 40A through 40C, the clamps 174A and 174B are opened, and the handle of the medical device placed in the clamps, with the actuator ring of the medical device aligned with clamp 174A, and the remainder of the handle aligned with clamp 1748. The covers 264 of the clamps 174A and 174B are closed, and the latches 270 engaged.

Once the medical device is engaged in the remote manipulator system, the distal end of the medical device can be introduced into the body and manipulated with the remote manipulation system 50.

When it is desired to advance the catheter, the drive 148 is actuated which causes the translation mechanism 212 to advance the body 170 of the device driver 52 relative to the base 160, which advances the catheter mounted thereon. When it is desired to retract the catheter, the drive 148 is actuated which causes the translation mechanism 212 to retract the body 170 of the device driver relative to its base 160. When it is desired to rotate the distal end of the catheter, the drives 142 and 146 are operated to operate transmissions 206 and 310 which cause the splines 230 of both of the clamps 174A and 174B to turn, thereby turning the split adapter ring gears 302 and thus, the entire device engaged therein. When it is desired to operate the actuation ring on the handle of the device, relative translation of the ring and the remainder of the handle can be caused by operating the drive 144 to operate the translation mechanism 208 to cause the clamp 174B engaging the handle to move relative to the clamp 174A engaging the actuator ring, to thereby cause relative movement between the actuator ring and the handle. Relative rotation of the actuator ring and the remainder of the handle can be caused by operating the drive 142 to operate transmission 206 to operate the spline 230 of clamp 174A, rotating the actuator ring relative to the remainder of the handle, or operating drive 210 to operate transmission 210 to operate the spline 230 of clamp 174B rotating the remainder of the handle relative to the actuator ring, or to operate drives 142 and 146 and different rates and or in different directions to cause relative rotation between the actuator ring engaged in clamp 174A and the remainder of the handle engaged in 174B.

The drives 142, 144, 146, and 148 can be under direct control by a physician through a suitable interface, or the drives can be under the control of a microprocessor under the supervision and direction of a physician.

In an emergency, the clamps 174A and 174B can be easily opened by operating latches 270 to release the cover and pulling the medical device free. The split adapter rings can be easily removed from the device so that the device can be used manually.

Embodiments of the remote manipulator device can be adapted to a wide variety of medical devices to allow the devices to be positioned and operated inside the body under remote control by a physician, or by physician-supervised computer control.

The foregoing description of the embodiments has been provided for purposes of illustration and description. It is not intended to be exhaustive or to limit the invention. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the invention, and all such modifications are intended to be included within the scope of the invention.

What is claimed is:

1. A system for operating a catheter having a distal end adapted to be navigated in the body, and a proximal end having a handle with a translatable control and a rotatable control for acting on the distal end of the device, the system comprising:
a support for receiving and engaging the handle of the catheter;
a translation mechanism for advancing and retracting the support to advance and retract a catheter whose handle is received in the support;
a rotation mechanism for rotating the support to rotate a catheter whose handle is received in the support;
a translation operator for engaging the translatable control of a catheter whose handle is received in the support and operating the translatable control to act on the distal end of the device; and
a rotation operator for engaging the rotatable control of a catheter whose handle is received in the support and operating the rotatable control to act on the distal end of the device.

2. The system according to claim 1 wherein the rotation mechanism and the rotation operator operate in a coordinated manner to rotate the catheter without operating the rotatable controller.

3. The system according to claim 1 wherein the rotation mechanism comprises a rotatable clamp for receiving the handle of a catheter.

4. The system according to claim 3 wherein the rotation operator comprises a rotatable clamp for receiving the rotation control of a catheter.

5. In combination with a catheter having a distal end adapted to be navigated in the body, and a proximal end having a handle with a translatable control and a rotatable control for acting on the distal end of the device, a system for remotely manipulating the catheter, the system comprising:
a support for receiving and engaging the handle of the catheter;
a translation mechanism for advancing and retracting the support to advance and retract a catheter;
a rotation mechanism for rotating the support to rotate the catheter;
a translation operator for engaging the translatable control of the catheter and operating the translatable control to act on the distal end of the device; and
a rotation operator for engaging the rotatable control of the catheter and operating the rotatable control to act on the distal end of the device.

6. A system for remotely manipulating an elongate medical device of the type having a distal end adapted to be introduced into a patient and a handle at the proximal end with rotatable and translatable controls for manipulating the device, the system comprising:
a device driver positionable adjacent the patient;
a device interface releasably mounted on the device driver, the device interface comprising a first clamp for releasably engaging a first portion of the handle of the medical device and a second clamp for releasably engaging a second portion of the handle of the medical device; and
a control for selectively operating the device driver to advance and retract the device driver relative to the patient to advance and retract the distal end of the medical device in the patient; selectively operating the device driver to cause the first and second clamps to rotate the first and second portions of the handle of the medical device to rotate the distal end of the medical device in the patient;
selectively operating the device driver to move the second clamp relative to the first clamp to cause relative translation between the first portion of the handle and the second portion of the handle to thereby operate the translatable control on the handle; and selectively operating the device driver to cause the at least one of the first and second clamps to rotate the portion of the medical device releasably engaged therein, to cause relative rotation between the first portion of the handle and the second portion of the handle to thereby operate the rotatable control on the handle.

7. The system according to claim 6 wherein each of the first and second clamps comprises a base having a concave recess, a hinged cover having a concave recess, the cover being pivotally operable between an open position in which a portion of the handle of a medical device can be inserted and removed from the clamp, and a closed position in which the concave recesses in the base and cover cooperate to create a central opening for receiving and retaining the portion of the handle of the medical device inserted therein; and a latch for releasably securing the cover in its closed position.

8. The system according to claim 7 wherein each of the clamps further comprises an adapter ring, ratably mountable in the central opening between the cover and the base, and comprise first and second hingedly connected portions adapted to enclose a portion of the handle of the medical device.

9. The system according to claim 8 wherein the adapter ring includes a ring gear, and wherein the base includes a gear train engaging the ring gear on the adapter ring and engagable with the device driver to turn the adapter ring within the clamp.

10. The system according to claim 6 wherein the device interface comprises a tray, a first clamp mounted on the tray in a fixed position, and a second clamp slidably mounted on the tray.

11. The system according to claim 10 wherein the first and second clamps have portions extending through the tray, adapted to engage portions of the device driver.

12. A system for remotely manipulating an elongate medical device of the type having a distal end adapted to be introduced into a patient and a handle at the proximal end with rotatable and translatable controls for manipulating the device, the system comprising:
a device driver positionable adjacent the patient;
a device interface releasably mounted on the device driver, the device interface comprising a tray having a first clamp for releasably engaging a first portion of the handle of the medical device in a rotatable adapter ring and a second clamp slidably mounted on the tray, for releasably engaging a second portion of the handle of the medical device in a rotatable adapter ring; each of the clamps including a portion extending through the tray and engaging the device driver, and a gear train engagable with the device driver for rotating the adapter ring; and
a control for selectively operating the device driver to advance and retract the device driver relative to the patient to advance and retract the distal end of the medical device in the patient; selectively operating the device driver to cause the first and second clamps to rotate the first and second portions of the handle of the medical device to rotate the distal end of the medical device in the patient; selectively operating the device driver to move the second clamp relative to the first clamp to cause relative translation between the first portion of the handle and the second portion of the handle to thereby operate the translatable control on the handle; and selectively operating the device driver to cause the at least one of the first and second clamps to rotate the portion of the medical device releasably engaged therein, to cause relative rotation between the first portion of the handle and the second portion of the handle to thereby operate the rotatable control on the handle.

13. The system according to claim 12 wherein the device driver comprises a base; a body, a translation mechanism for translating the body relative to the base; a transmission for engaging a portion of the first clamp mounted thereon to operate the gear train to rotate the adapter ring of the first clamp; a translation mechanism engaging a portion of the second clamp for translating the second clamp relative to the first clamp; and a transmission for engaging a portion of the second clamp mounted thereon to operate the gear train to rotate the adapter ring of the second clamp.

14. A device driver for mounting and operating a device interface to remotely manipulate an elongate medical device engaged in the device interface, the medical device of the type having a distal end adapted to be introduced into a patient and a handle at the proximal end with rotatable and translatable controls for manipulating the medical device, the device driver comprising:
a base; and
a body slidably mounted on the base; a translation mechanism in the body for translating the body relative to the base; a socket that receives and selectively rotates a portion of a first part of the device interface, a socket that receives and selectively translates a portion of a second part of the device interface; and a socket that receives and selectively rotates a portion of the second part of the device interface.

15. A device interface for mounting on a device driver to engage and operate a medical device of the type having a distal end adapted to be introduced into a patient and a handle at the proximal end with rotatable and translatable controls for manipulating the medical device, the device interface comprising:
a tray;
a first clamp mounted on the tray, the first clamp comprising a base, a hinged cover; and a split adapter ring, rotatably mounted in the base and cover, adapted to receive a first portion of the handle of the medical device, and a gear train for rotating the split adapter ring, the gear train having a projecting spline adapted to engage a socket on a device driver on which the device interface is mounted so that the device driver can rotate the split ring adapter of the first clamp; and
a second clamp slidably mounted on the tray, the second clamp comprising a base, a hinged cover; and a split adapter ring, rotatably mounted in the base and cover, adapted to receive a second portion of the handle of the medical device, and a gear train for rotating the split adapter ring, the gear train having a projecting spline adapted to engage a socket on a device driver on which the device interface is mounted so that the device driver can rotate the split ring adapter of the second clamp, the second clamp having a depending portion adapted to engage a translation mechanism on a device driver on which the device interface is mounted so that the device driver can translate the second clamp relative to the first clamp.

16. A method of operating a medical device of the type having a distal end adapted to be introduced into a patient and a handle at the proximal end with rotatable and translatable controls for manipulating the device, the method comprising:
engaging a first portion of the handle of the medical device in an adapter ring releasably rotatably mounted in a first clamp;
engaging a second portion of the handle of the medical device in an adapter ring releasably rotatably mounted in a first clamp; and
selectively moving the first and second clamps together to advance and retract the distal end of the medical device in the patient; selectively rotating the adapter rings of the first and second clamps to rotate the distal end of the medical device in the patient; selectively moving the second clamp relative to the first clamp to cause relative translation between the first portion of the handle and the second portion of the handle to thereby operate the translatable control on the handle; and selectively differentially rotating the adapter rings of the first and second clamps to thereby operate the rotatable control on the handle.
